(19) United States
(12) Patent Application Publication
(10) Pub. No.: US 2003/0173220 A1
(43) Pub. Date: Sep. 18, 2003

(54) Charged carrier material and its use

(76) Inventors: Bengt Bjellqvist, Uppsala (SE); Ronnie Palmgren, Uppsala (SE)

Correspondence Address: AMERSHAM BIOSCIENCES, PATENT DEPARTMENT, 800 CENTENNIAL AVENUE, PISCATAWAY, NJ, 08855, US

(21) Appl. No.: 10/380,081

(22) PCT Filed: Sep. 12, 2001
(86) PCT No.: PCT/EP01/10512

(30) Foreign Application Priority Data
Sep. 19, 2000 (SE) 0003358-9

(51) Int. Cl. G01N 33/559

(52) U.S. Cl. 204/456; 204/469

(57) ABSTRACT

An electrophoresis separation material, especially for isoelectric focusing, comprising a carrier material to which pH-buffering groups are firmly attached and anode side and cathode sides. At least a first portion of the pH-buffering groups (a) having a pH-dependent charge on a nitrogen atom binding to an sp2-hybridised carbon atom,(b) including groups with pKa≧9.5. One embodiment, the groups in (a) have the structure —(NH)ₙC(═NH)(NH2)(I), possibly in protonated form, where n is 0 or 1, and the free valence binds to the carrier material via an organic linker. An IEF separation material obtained by polymerisation of a mixture of monomers and containing immobilised buffering groups capable of establishing an immobilised pH-gradient where(1) at least one monomer has the structure CH2═CHR″CONR₁₂R₁₃ (V) where (i) R″ represents hydrogen or methyl; (ii) one or more of R₁₂ and R₁₃ are —((CH₂)ₙ′—O)ₙ″—H where n′ is 2-3 and n″ is 1-5 while any remaining group R₁₂ and R₁₃, is hydrogen and when n is 3, a hydrogen on the middle carbon in (CH₂)ₙ may be replaced with hydroxy; and (2) the immobilised pH gradient extends to pH>10.

### TECHNICAL FIELD

The present invention relates to a separation material, which can be used when separating amphoteric compounds based on differences in isoelectric points (pI), in particular by electrophoresis (isoelectric focusing, IEF).

Electrophoresis encompasses separation methods in which charged or chargeable solutes are separated from each other by means of mass transport in a liquid, primarily aqueous, caused by an applied electric field. In order to minimise disturbances caused by convection the liquid typically is contained within a porous carrier material (gel electrophoresis), or in capillaries (capillary electrophoresis, CE) or in channels segmented by porous membranes across the direction of mass trasport In the context of the invention the term carrier material shall encompass also the walls of the capillaries, the channels and porous membranes just mentioned in/through which solutes are intended to pass.

The substances separated are typically bio-organic and encompass primarily compounds having polypeptide structure and/or carbohydrate structure. Proteins are particularly important

### BACKGROUND

In certain kinds of electrophoresis the carrier material has been functionalized with groups which provide conditions that are beneficial for the intended separation. One important kind of groups has been pH-buffering groups. By immobilising pH-buffering groups of different pKa's between the anode end and the cathode end of an electrophoretic gel it became possible during the late seventies to set up immobilised pH-gradients (Aminkemi, U.S. Pat. No. 4,130,470) to be used in isoelectric focusing. In order to have good pH-gradients it was important to have a range of different pH-buffering groups with increasing/decreasing pKa values spaced within a desired pH-interval. The difference between the pKa of two neighbouring buffering groups has typically been 1-2 pH units. For pH intervals extending above pH 10 there has in principle been available only groups that are hydroxide forms of quaternary ammonium groups, i.e. groups that by themselves lack pKa values and buffering capacity and have a pH-independent charge. For pH>10.5, the recognised acid/base pair thus has been H₂O/OH⁻. There has been a demand to have access to improved buffering groups, which has higher pKa values than tertiary ammonium groups.

pH-gradients extending above pH 10 have not had the sufficient stability and quality for permitting focusing during the sufficient time required for a high-quality result There has thus also been a demand for improved combinations of carrier materials and buffering groups with pKa higher than pKa of tertiary ammonium groups in order to lower the risk for hydrolysis at pH>10.

Immobilised pH gradients have been described by Chiari et al (J. Chromatog. 559 (1991) 119-131), Chiari et al (J. Chromatog. 548 (1991) 381-392), Chiari et al (Applied and Theoretical Electrophoresis 1 (1989) 99-102 and 103-107). The problems with isoelectric focusing at pH extremes have been described (Mosher et al Electrophoresis 7 (1986) 59-66).

In a number of scientific articles, there have been described groups containing the structure

—Ar—C(═NH)(NH₂)

possibly in protonated form as ligand in affinity chromatography for the separation of various serine proteases. Ar is an aryl moiety. See Chang et al (J. Chem. Tech, Biotechnol. 59 (1994) 133-139); Lee et al (J. Chromatog. A 704 (1995) 307-314); Kharnlichi et al., J. Chromatog. 510 (1990) 123-132; and Burton et al (U.S. Pat. No. 5,789,578). The same group has also been described in SE patent application 9904197-2 (Amersham Pharmacia Biotech AB) filed Nov. 22, 1999. In this latter case the group is used as a nixed mode ion exchange ligand for the separation of proteins by ion exchange adsorption.

Acrylamide monomers of the structures:

CH₂═CH(CH₃)CONHC(═NH)(NH₂) or

CH₂═CH(CH₃)CONH(CH₂)₃NHC(═NH)(NH₂)

have been used for the synthesis of imprinted macroporous polymers in which this kind of monomers provides basic functional groups. These polymers have been tested for binding acidic molecules in column chromatography experiments See Spivak et al., J. Org. Chem 64 (1994) 4627-4634.

U.S. Pat. No. 5,055,517 (Shorr et al), U.S. Pat. No. 5,219,923 (Shorr et al), U.S. Pat. No. 5,438,092 (Kozulic), U.S. Pat. No. 5,066,376 (Osterhoudt et al), WO 93 11174 (Righetti et al), WO 9716462 (Righetti), and WO 9810276 (Kozulic), all of which hereby are incorporated by reference, describe carrier material for electrophoresis based on N-alkyl substituted amides and/or alkyl esters of acrylic and/or methacrylic acids. The alkyl groups have contained also hydroxy and ether groups to provide a sufficient hydrophilicity to the ready-made gel. In particular WO 9311174 (Righetti et al) and WO 9716462 (Righetti) gives carrier materials that has been treated in alkaline milieu and subsequently used in isoelectric focusing with soluble as well as immobilised pH gradients extending up to pH=10. See also Simò-Alfonso et al., Electrophoresis 17 (1996) 723-731 and 17 (1996) 732-737 and Gelfi et al., Electrophoresis 17 (1996) 738-743. A review of new types of polymers for electrophoresis available 1995 has been published by Chiari et al (Electrophoresis 16 (1995) 1815-1829).

Objectives of the Invention

A first objective is to provide improved separation material that can be used for isoelectric focusing at alkaline pH>10.0 under sufficient time for providing high quality results, e.g. without significantly destabilising a pH gradient used at these pH values.

A second objective is to provide a separation material, including a pH-gradient separation material, comprising a carrier material which is functionalized with buffering groups with pKa values above 10.0, eg. ≧10.5 or ≧11.0, and which has an improved stability and an optimal hydrophilicity in this pH range.

This objective concerns primarily separation materials in form of porous gels, capillaries or channels with porous membranes as described above. The intended use is for the kind of methods given for the third objective.

A third objective is to provide improved electrophoretic separation methods that involve separations at pH>9, e.g. >pH 10 or pH>11, and the presence of pH-buffering groups that are immobilised to a carrier material. Primarily the methods involve isoelectric focusing (IEF) with immobilised pH gradients of compounds having pIs>9.5, e.g. ≧10 or ≧10.5.

A fourth objective is to provide new buffering groups and monomers that have a pKa>10.0, e.g. ≧10.5 or ≧11.0, and an improved stability against hydrolysis at pH>10 for the manufacture of separation materials as discussed above.

### THE PRESENT INVENTION

The present inventors have recognised that these objectives can be at least partially met by

(1) properly selecting immobilised pH-buffering groups amongst those that have

(a) a pH-dependent charge on a nitrogen atom which binds to an sp²-hybridised carbon atom, and

(b) a pKa≧9.5, e.g. ≧10.0 or ≧10.5 or ≧11.0, and/or

(2) utilising carrier materials, which have been based on certain acryl monomers. pH-buffering groups complying with la will in the context of the invention be called M-groups. Typical M-groups are according to formula I:

—(NH)ₙC(═NH)(NH₂) (I)

Individual groups of formula (I) may be in protonated (charged) form or in unprotonated (uncharged) form. The anchoring of the groups to the carrier material may be via a linker. n is an integer 0 or 1. The hydrogens can be replaced as discussed below.

The First Aspect. Separation Materials having M-groups with pKa>9.5.

This aspect concerns an electrophoresis separation material comprising a carrier material and a plurality of pH-buffering groups firmly attached to a carrier material via a linker. The main characterising feature of this aspect is that at least a portion of the plurality are M-groups and have a pKa≧9.5, e.g. ≧10.0 or ≧10.5 or ≧11.0. A typical upper limit for suitable pKa values corresponds to the pKa of water. Preferred groups are selected amongst those with formula (I).

pKa values referred to here and henceforth are measured in aqueous solutions (25° C.) for a low molecular compound/monomer which has the particular M-group concerned and at least a representative part of the linker binding to the base skeleton of the carrier material. The pKa values have been extrapolated to ionic is strength zero. See Handbook of Chemistry and Physics, 56^{th} edition (1975-1976) page D-133, CRC Press, 18901 Cranwood Parkway, Cleveland, Ohio, U.S.A.

There may be present one, two, three or more different kinds of M-groups. At least one kind of the different M-groups has a pKa as discussed above.

The compound of formula I also includes also included that at least one of the hydrogens in —(NH)ₙC(═NH)(NH₂) can be replaced with a straight, branched or cyclic hydrocarbon group (R), e.g. alkyl group, that may be equal or different for different hydrogens. Typical hydrocarbon groups comprise up to 10 carbon atoms. In the hydrocarbon group, the carbon chain may be interrupted at one or more positions by an ether oxygen (—O—), an amino nitrogen (—NR₁—) or a thioether sulphur

(—S—), in particular an ether oxygen. The expression hydrocarbon group and alkyl group includes that a hydrogen at one or more positions may be replaced with a group selected amongst —OR₁, —NR₂R₃, or —SR₄, in particular —OR₁. Each of R₁-R₃ may independently represent hydrogen, or a lower straight, branched or cyclic clic alkyl group, typically a hydrogen and/or C₁₋₃ alky. R₄ is selected in the same manner as R₁-R₃ except that alkyls are preferred. For hydrocarbon groups, such as alkyl groups, which have more than 2-3 carbons, the ratio of the number of heteroatoms (in particular oxygens) to the number of carbon atoms should be in the interval 0.25-0.80. By the term “heteroatoms” is meant oxygen, nitrogen and sulphur, in particular oxygen. In preferred variants one and the same sp³-hybridised carbon atom in an alkyl group carries at most one single-bound heteroatom. The hydrogens may also be replaced in a pair-wise manner with a bivalent C₂₋₃ hydrocarbon group possibly providing double bonds conjugated with the C═NH group in formula (I).

The linker provides covalent attachment of a group of formula I to the carrier material. Typically the linker has an sp³-hybridised carbon atom or an aromatic carbon atom directly attached to the free valence in the group represented by formula (I). The preference is for an sp³-hybridised carbon atom because an aryl group may easily give rise to unwanted interactions with various bioorganic solutes in the aqueous liquid containing the substances to be separated.

The linker stretches from the basic skeleton of the carrier material to the group of formula I and may thus also comprise parts of the carrier material projecting from its base skeleton. The linker is preferably uncharged. Thus in the preferred variants the linker may comprise structures selected from straight, branched or cyclic bivalent hydrocarbon groups possibly being substituted with —OR₅, or —SR₆, in particular —OR₇. The linker may also comprise ether groups (—O—), thioether groups

(—S—), amido groups (—CONR₈—), ester groups (—CO—O—) and/or other uncharged groups of similar or higher stability against hydrolytic and/or oxidative cleavage. R₅-R₈ are selected among hydrogen and the same groups as R. Alkyls are preferred for R₆. For stability reasons there is often at most one single bond to a heteroatom from one and the same sp³-hybridised carbon atom. Preferably the linker is an alkylene chain, possibly interrupted at one or more positions by ether oxygen (—O—) and/or possibly substituted at one or more positions with a hydroxy group. The linker typically has a length of ≦20 atoms, such as ≦10 atoms.

Illustrative examples of preferred linkers are according to formulae II:

—CO—NR₉—[(CH₂)ₘO]ₖ(CH₂)_{m′}— (II)

where R₉ is selected amongst hydrogen and the same groups as R, m and m′ are integers selected in the interval 2-10, such as 2, 3 and 4, and k is an integer 0 or larger, such as less than 7. The left free valence typically binds to the base skeleton of the carrier material and the right free valence binds to the group of formula I, or vice versa. Particularly interesting linkers of this kind provide a chain of 3-8 atoms for linking the group of formula I to the base skeleton of the carrier material. The group —NR₉— may be replaced with —O—.

The Carrier Material and the Manufacture of the Carrier Material Functionalized with Groups of Formula I

The carrier material may be based on a native or a synthetic polymer.

Illustrative synthetic polymers are typically cross-linked and based on unsubstituted and/or N-alkyl substituted acrylamides or methacrylamides, N-vinyl substituted saturated carboxamides including such formamides, alkyl esters of acrylic or methacrylic acids, vinyl aryls and other monomers comprising polymerisable unsaturated groups such as various vinyl groups. By including corresponding monomers exhibiting two or more polymerisable unsaturated groups in the polymerisation mixtures, the obtained polymer will be cross-linked. For monomers containing two or more methyl groups and/or alkyl groups with more than 2-3 carbon atoms, the methyl/alkyl group preferably should show a pronounced hydrophilicity. This will be accomplished if methyl/alkyl groups are functionalized with one or more of hydroxy, ether, tihioether groups and the like to an extent giving a ratio of heteroatoms (as defined above, in particular oxygens) to carbon atoms in the interval 0.25-0.80.

One can envisage advantages if the polymerisation mixture contains monomers of formula m and/or corresponding bis- tris- etc forms:

CH₂═CR′—CO—X—R₁₀ (III)

X may be —O— (ester oxygen) or —NR₁₁—(N is an amido nitrogen) or nothing. R′ is hydrogen or methyl. R₁₀ and R₁₁ are selected from hydrogen and the alkyl groups defined for R. If X is an ester oxygen (—O—), R₁₀ preferably shall contain one or more hydroxy or ether oxygen as defined for R. If X is —NR₁₁—, one or both of R₁₀ and R₁₁ preferably shall contain one or more hydroxy or ether oxygen as defined for R, with the remaining group of R₁₀ and R₁₁, if any, preferably shall be hydrogen. Other polymerisable monomers containing one, two or more polymerisable unsaturations may be included in the polymerisation mixture, e.g. other acryl and/or methacryl monomers including their bis-, tris- etc forms.

If monomers of formula III and/or corresponding bis- tris- etc forms are present in the polymerisation mixture, their total amount typically is above 5 mol-% of the total amount of polymerisable monomers.

Monomers of formula III and bis-, tris- etc forms thereof, and their advantages and manufacture have been extensively deseribed in for instance U.S. Pat. No. 5,055,517 (Shorr et al), U.S. Pat. No. 5,219,923 (Shorr et al), U.S. Pat. No. 5,438,092 (Kozulic), U.S. Pat. No. 5,066,376 (Osterhoudt et al), WO 93 11174 (Righetti et al), WO 9716462 (Righetti), and WO 9810276 (Kozulic), all of which hereby are incorporated by reference. For a review of the optimal gels available 1995 see Chiari et al (Electrophoresis 16 (1995) 1815-1829).

The monomer may be polymeric as such (prepolymer) and carry a plurality of unsaturated structures. Typical prepolymers are selected among polyhydroxy polymers, such as dextran, agarose and other polysaccharides. See U.S. Pat. No. 4,094,832; U.S. Pat. No. 4,094,833; EP 87995; WO 9731026, WO 9726071, all of which hereby are incorporated by reference.

Another kind of synthetic polymers that can be used as carrier material is based on condensation polymers in which the monomers are selected from compounds exhibiting two or more groups selected among amino, hydroxy, carboxy etc groups. This kind includes polyamides, polyamines, polyethers etc. The base skeleton is formed by the carbon chains and the amine, amide, ether groups formed during polymerisation.

Illustrative examples of useful native polymers are cellulose, agarose, dextran, polyvinyl alcohol etc. Each of these polymers may be cross-linked to give an appropriate rigidity.

The carrier material may be in form of a flat bed gel. The carrier material may also be the interior wall of a capillary, for instance in form of a gel attached to a naked form of the interior wall of the capillary. See also above under the heading “Technical Field”.

The electrophoresis separation materials of the invention have a cathode side/end and an anode side/end at the opposite side. The immobilised M-groups may be present all throughout the carrier material between these two sides. Their concentration may be evenly distributed or there may be a higher concentration at the cathode side compared to the anode side, for instance in form of an increasing concentration gradient

In the preferred variants of the first aspect of the invention, the separation material comprises an immobilised pH-gradient encompassing at least the pH-interval 10-13 or parts thereof, such as 11-13, 10-12 etc. The characteristic feature of this variant is that M-groups assist in defining the pH-interval 10-13 or a part thereof, such as 10-11, 10-12, 11-13 etc. The interval 10-13 or parts thereof may be part of a larger interval that in turn may start in the interval of pH 2-3, 3-4, 4-5, 5-6, 6-7, 7-8, 8-9, 9-10, 10-11 or 11-12 and end in the interval of pH 11-12, 12-13, 13-14 or above. Typically the pH-gradient covers at least 1-2, preferably at least 2-3, pH units. The M-group has a pKa≧9.5, e.g. ≧10.0 or ≧10.5 or ≧11.0 and can for instance be according to formula I.

Depending on the width of the pH-gradient there may also be included one or more additional pH-buffering groups having pKa(s) below the pKa of an M-group which is present in the separation material and which has a pKa≧9.5, e.g ≧10.0 or ≧10.5 or ≧11.0. These additional groups are selected according to principles generally known in the field. See the publications discussed above relating to immobilised pH gradients

In an alternative variant the carrier material is a channel that is segmented by pH-buffering porous membranes placed across the channel such that there is an increasing/decreasing pH gradient along the length axis of the channel. One end of the channel is the anode end and the other the cathode end. For each segment, the cathode end membrane often has a pH-buffering groups of higher pKa than the anode end membrane.

M-groups, for instance of formula I, may be introduced onto a ready-made carrier material by reacting an at least bifunctional compound exhibiting both

(a) an M-group, in particular of formula I, or a group transformable to such a group, and

(b) a reactive group capable of forming covalent bonds with a reactive group in the ready-made carrier material, for instance a nucleophilic group in the bifunctional compound if the carrier material comprises an electrophilic group, or vice versa.

The M-groups have pKa≧9.5, e.g. ≧10.0 or ≧10.5 or ≧11.0.

Bifunctional reagents of this kind for funtionalizing carrier materials for chromatography have been described in SE patent application 9904197-2 (Amersham Pharmacia Biotech AB) and in U.S. Pat. No. 5,789,578 (Burton et al). The analogous approach can be utilised in funtionalizing carrier materials for electrophoresis.

It is often simpler to accomplish satisfactory separation materials according to the invention by including a polymerisable monomer exhibiting an M-group, in particular of formula I, in a polymerisation mixture containing other monomers as defined above and then initiate the polymerisation. Monomers exhibiting an M-group typically comprise the following moieties:

A. An M-group, for instance according to formula I, and with a pKa≧9.5, e.g. ≧10.0 or ≧10.5 or ≧11.0;

B. A polymerisable carbon-carbon or carbon-carbon triple double bond as discussed above for the monomers used to prepare the carrier material; and

C. A bridge structure attaching A to B and comprising structures selected in the same manner as for the linker. See above.

Monomers of this kind can be synthesised as outlined in the background art. See Spivak et al., J. Org. Chem. 64 (1999) 4627-4634. See also the experimental part.

When preparing the separation materials, e.g. gels, containing an immobilised pH-gradient, one may start from a first polymerisation solution having a pH corresponding to the lower (acidic) end of the gradient and a second polymerisation solution having a pH corresponding to the upper (alkaline) end of the gradient One or both of the solutions contain polymerisable monomers, including a range of monomers having pH-buffering groups of increasing pKa, at least one of which is an M-group with a pKa≧9.5 e.g. ≧10.0 or ≧10.5 or ≧11.0, and in particular is according to formula I. By gradient mixing the two solutions and applying the appropriate initiating system, carrier materials containing an immobilised pH-gradient can be obtained in analogy with the manufacture of gels containing a cross-linking gradient Typically the carrier material is cast in an appropriate cassette by filling the cassette with the appropriate gradient mixture of the two solutions as known in the art (an increasing amount of one of the solutions into the other) and polymerise. Se textbooks such as Reiner Westmeier, “Electrophoresis in Practice: A Guide to Theory and Practice”, VCH Verlagsgeschellschaft GmbH, Weiheim, Germany (1993, English version), in particular pages 197-220).

Capillaries containing immobilised pH gradients can be produced by

(a) providing a solution containing monomers of increasing/decreasing pKas and if needed an appropriate initiator;

(b) providing a capillary which has walls to which the monomers can be grafted and filling the capillary with the solution from step (a);

(c) applying an electric field along the length axis of the capillary to establish a pH gradient along the axis; and

(d) initiating grafting as known in the field.

Finally the remaining solution is removed and the capillary conditioned for electrophoresis and/or storage.

The principles for capillaries outlined above have general applicability. The principles are thus also useful for the production of isoelectric focusing separation materials containing only buffering groups with pKa<11.0, e.g. <10.0, and/or quaternary groups that is or can be transformed to their hydroxide form by ion exchange.

As known in the art, the polymerisation/grafting systems applied in the present invention can make use of various kinds of initiating systems that may or may not include an initiator. Thermal and chemical initiators are often used to initiate polymerisation. Thermal initiators are often preferred. They have their best efficiency in the range of 50-90° C. Well-known chemical/thermal initiators are azo compounds (for instance 2,2′-azobis2,4-diethylvaleronitrile), azoisonitriles, peroxides (for instance benzoylperoxide), persulphates. One important kind of chemical initiators requires irradiation, for instance UV, in order to start the polymerisation. Redox systems have also been used, for instance Fenton's reagent (hydrogen peroxide +Fe²⁺). Initiation of polymerisation or grafting may take place without initiators, for instance by electron beam irradiation or γ-irradiation.

The Second Aspect. Separation Material Containing M-groups of Optional pKa.

This aspect concerns an isoelectric focusing separation material containing an inrobilised pH gradient. This aspect is characterised in that the separation material comprises pH-buffering groups, which are M-groups for instance of formula I, for defining at least a part of the pH interval of the gradient. In advantageous variants of this aspect, the gradient/interval extends to pH>10 as discussed for the first aspect of the invention with at least one kind of M-groups with pKa≧9.5, e.g. ≧10.0 or ≧10.5 or ≧1 1.0, being included. In other respects the second aspect encompasses the various embodiments outlined for the first aspect of the invention.

Third Aspect. The Use the Inventive Separation Material

This aspect concerns a method for performing electrophoresis in a separation material having the features defined above. This aspect in particular concerns isoelectric focusing in an immobilised pH gradient comprising at least a pH-interval as defined above. The method comprises the steps of (a) applying to the separation material a sample containing the substances to be separated from each other, and (b) applying an electrical potential over the pH gradient for a sufficient time for the substances to separate from each other according to their pI. Subsequently the individual substances differing in pI can be identified and/or subjected to further separation steps and/or subjected to chemical derivatization and/or collected and/or analysed. Isoelectric focusing is often followed by gel electrophoresis in which each separated substance is further separated according to molecular weight and/or molecular size. Isoelectric focusing and gel electrophoresis is typically run perpendicular two,, each other (2-dimensional gel electrophoresis).

Typical isoelectric focusing is performed for at least 4-5 hours or more in order to accomplish a sufficient resolution, i.e. for separating amphoteric compounds differing in pI with 0.01 or more. The focusing time thus can be 6-7 h or more including overnight focusing.

The Fourth Aspect. The use of Polymerisable Momomers having M-groups.

This aspect concerns the use of a polymerisable monomer exhibiting an M-group as defined above for the manufacture of a separation gel as described above. The M-group is in particular according to formula I and/or has pKa as defined above.

The Fifth Aspect. Isoelectric focusing Material Containing Immobiliness and Suitable for pH>10.

This aspect concerns an isoelectric focusing separation material, preferably in gel form, containing immobilised buffering groups defining an immobilised pH gradient A characteristic feature of this separation material is that it is obtained by polymerising a mixture of monomers which includes a monomer according to formula III above (monomer 1) and that the pH gradient extends to pH values >10, e.g. ≧10.5 or ≧1 1.0. In preferred variants monomer I and bis-, tris- etc forms thereof constitute a significant part such as ≧5 mol-% of the polymerisable monomers of the mixture. In other preferred variants monomer 1 complies with formula IV:

CH₂═CHR″CONR₁₂R₁₃ (IV)

R″ represents hydrogen and methyl. R₁₂ and R₁₃ have the same meaning as R₁₀ and R₁₁ above, with the further preference that one or more of them are —((CH₂)_{n′}—O) _{n″}—H where n′ is an integer 2-3 and n″ is an integer 1-5 while the remaining group of R₁₂ and R₁₃, if any, is hydrogen. Preferred values of n and n′ are 3 and 1, respectively. On carbon atoms, which bind no ether oxygen, one hydrogen atom may be replaced with a hydroxy group. Hydrogens bound to an sp³-hybridised carbon may be replaced with hydroxy methyl.

The polymerisation mixture used for the manufacture of the separation material of the fourth aspect may also include one or more polymerisable monomers having other structures than according to formula III or IV as discussed above for the first aspect of the invention. Typical other monomers are other acrylamides 5 and other methacrylamides including bis-, tris- etc forms thereof

The separation material of this fourth aspect of the invention preferably contains a plurality of pH buffering group with pKa>10.0, ≧10.5 or ≧11.0, which may or may not include quaternary ammonium groups in hydroxide form, for defining the part of the gradient extending to pH>10, e.g. ≧11.0. Preferably a portion of the plurality of pH-buffering groups are M-groups with the appropriate pKa values.

The pH-buffering group with pKa≧10.0, e.g. ≧10.5 or ≧11.0, may have been introduced by including a polymerisable monomer having this kind of group in the polymerisation mixture.

The pH gradient of the separation material according to the fourth aspect may be of the same kind as described above for the first aspect. The separation material also has a cathode and an anode side as well known in the field and described in the context of the first aspect of the invention. The separation material of the fourth aspect of the invention may be in any of the forms discussed above for the other aspects of the invention.

In the various aspects of the invention it is believed that the optimal carrier materials should be based on acrylamide monomers as defined in formula In, particularly according to formula IV. At the priority filing preliminary experiments had indicated the best results for carrier material based on CH₂═CH₂CONH(CH₂)₃OH.

The invention will now be illustrated with a number of non-limiting patent examples. The invention is defined in more details in the appending claims.

### EXPERIMENTAL PART

Synthesis of Acrylamidoagmatine

Agmatine sulphate was desalted with barium hydroxide in water, barium- sulphate, which was formed precipitated in water and was filtered off. Agmatine (50 mmol) was dissolved in methanol and diisoproylamine (55 mmol) was added, the solution was cooled to 0° C. with an ice/water bath. 60 mmol acryloylchloride was added over a period of time (30 min) while the solution was kept at 0° C. Then the reaction solution was allowed to reach room temperature over night. The solvent was evaporated and the product was purified with flash chromatography on a RPC-18 column with a water/methanol gradient.

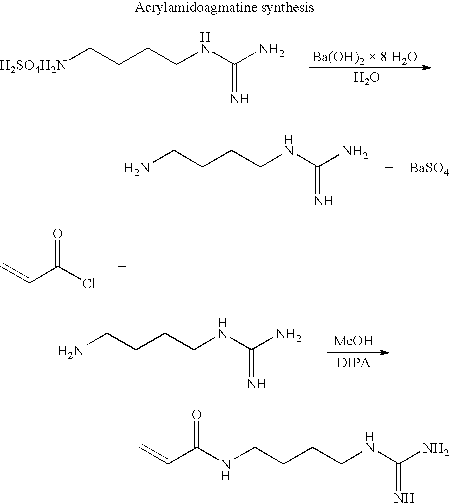

Polymerisation of Acrylamidoagmatine

Acrylamidoagmatine (AAA) was homopolymerised and copolymerised with acrylamide (AA) and acrylamidopropanol (AAP) in water, with APS (ammonium persulphate)Temed) redox system as initiator.

| Monomer | | [APS] | [Temed] | Water | AAA | AA | AAP |
|---|---|---|---|---|---|---|---|
| conc % | [M] | mM | mM | μl | ML | mL | mL |
| 6% | 0.83 | 3.25 | 4.5 | 1143.6 | 2.8 | 0 | 0 |
| 8% | 1.1 | 3.25 | 4.5 | 243.6 | 3.7 | 0 | 0 |
| 6% | 0.83 | 3.25 | 4.5 | 1143.6 | 1.4 | 1.4 | 0 |
| 8% | 1.1 | 3.25 | 4.5 | 243.6 | 1.85 | 1.85 | 0 |
| 6% | 0.83 | 3.25 | 4.5 | 1143.6 | 1.4 | 0 | 1.4 |
| 8% | 1.1 | 3.25 | 4.5 | 243.6 | 1.85 | 0 | 1.85 |

The copolymer made with AAP is much more stable against basic hydrolysis than the copolymer with AA

Crosslinking of Acrylamidoagmatine

A gel was made with acrylamidoagmatine (AAA) and acrylamide as monomer and bisacrylamide as a crosslinker. The system was polymerised in water with APS/Temed as initiator.

1. An electrophoresis separation material comprising a carrier material to which a plurality of pH-buffering groups are firmly attached and an anode side and a cathode side, characterised in that at least a first portion of the plurality are groups
(a) which have a pH-dependent charge on a nitrogen atom binding to an sp²-hybridised carbon atom, and
(b) which include groups with pKa≧9.5.

2. The material of claim 1, characterised in that the said groups of the first portion include groups that have the structure
—(NH)ₙC(═NH)(NH₂) (I)
possibly in protonated form, where n is 0 or 1, and the free valence binds to the carrier material via an organic linker.

3. The material of claim 2, characterised in that n is 1, and that the linker preferably provides an sp³-hybridised carbon atom directly attached to the free valence in the group of formula (I).

4. The material of any of claims 1-3, characterised in that said carrier material is the interior wall of a capillary or is a gel, possibly cross-linked, for instance in the form of a flat bed or a filled channel or capillary.

5. The material of any of claims 14, characterised in that a second portion of the plurality includes pH buffering groups having pKa-values different from the pKa(s) of the said groups in the first portion and that the pH-buffering groups in both the first and second portions are arranged to define an increasing pH-gradient going from the anode side to the cathode side.

6. The material of any of claims 1-5, characterised in that the carrier material is a vinyl polymerisate, for instance obtained by polymerisation of a polymerisation mixture which contains one or more monomers having the structure
CH₂═CR′—CO—X—R₁₀ (III)
in which
(a) R′ is hydrogen or methyl;
(b) X is an ester oxygen (—O—) or an amido nitrogen (—NR₁₁—) or nothing;
(c) at most one of R₁₀ and R₁₁ is selected from hydrogen and straight branched or cyclic C₁₋₁₀ alkyl groups each of which has a carbon chain that possibly is interrupted at one or more positions by an ether oxygen (—O—), an amino nitrogen (—NR₁—) or a thioether sulphur (—S—), in particular an ether oxygen, and/or a hydrogen possibly is replaced with a group selected among —OR₁, —NR₂R₃, or —SR₄, in particular —OR₁, where R₁-R₄ may independently represent hydrogen, or straight, branched or cyclic alkyl group;
with the proviso that when an allyl group has more than 2 carbons, the ratio of the number of heteroatoms (in particular oxygens) to the number of carbon atoms for the alkyl group is in the interval of 0.25-0.80.

7. The material of claim 6, characterised in that X-R₁₀ is —NR₁₀R₁₁— in which one or more of R₁₀ and R₁₁ are —((CH₂)_{n′}—O)_{n″}—H where n′ is an integer 2-3 and n″ is an integer 1-5 while the remaining group of R₁₀ and R₁₁, if any, is hydrogen,
with the proviso that when n′ is 3, a hydrogen bound to a carbon atom not binding a ether oxygen (—O—) in (CH₂)ₙ possibly is replaced with hydroxy.

8. An isoelectric focusing separation material, preferably in form of a gel, obtained by polymerisation of a mixture of monomers, and containing an immobilised pH-gradient defined by a plurality of pH-buffering groups differing in pKa, characterised in that
(a) at least one of the monomers has the structure
CH₂═CHR″CONR₁₂R₁₃ (IV)
in which
(i) R″ represents hydrogen or methyl;
(ii) one or more of R₁₂ and R₁₃ are —((CH₂)_{n′}—O)_{n″}H where n′ is an integer 2-3 and n″ is an integer 1-5 while the remaining group of R₁₂ and R₁₃, if any, is hydrogen,
with the proviso that when n′ is 3, a hydrogen bound to a carbon atom not binding a ether oxygen (—O—) in (CH₂)ₙ possibly is replaced with hydrogen; and
(b) the immobilised pH gradient extends to pH>10.

9. The separation material according to claim 8, characterised in that at least a portion of the plurality of different groups has a pH-dependent charge on a nitrogen atom binding to an sp²-hybridised carbon atom and a pKa≧9.5.

10. A method for performing electrophoresis, in particular isoelectric focusing, in a separation material comprising the steps of
(a) applying to the separation material a sample containing the substances to be separated from each other, and
(b) applying an electrical potential over the pH gradient for a sufficient time for the substances to separate from each other, characterised in that the separation material according to any of claims 1-9 is used.

11. The use of a polymerisable monomer comprising a charged or chargeable group (A), a polymerisable unsaturation (B), and a bridge structure (C) linking A to B, in which
A has a pH-dependent charge on a nitrogen atom binding to an sp²-hybridised carbon atom, in particular —(NH)ₙC(═NH)(NH₂) where n is 0 or 1 (Formula I);
B is a polymerisable carbon-carbon or carbon-carbon triple double bond; and C comprises structures selected from straight, branched or cyclic hydrocarbon chains possibly being substituted with hydroxy or lower alkoxy groups, ether oxygens, thioether sulphur, secondary or tertiary amino groups, amide groups etc.
for the synthesis of separation material for use in separation by electrophoresis, in particular isoelectric focusing.

12. The use according to claim 11 wherein the monomer has a pKa≧9.5, e.g. ≧10.0.
